(19) United States
(12) Patent Application Publication
(10) Pub. No.: US 2025/0249281 A1
(43) Pub. Date: Aug. 7, 2025

(54) Wearable Light Device With Dual Frequencies

(71) Applicant: Aleddra Inc., Renton, WA (US)

(72) Inventor: Chia-Yiu Maa, Bellevue, WA (US)

(21) Appl. No.: 19/186,404

(22) Filed: Apr. 22, 2025

Related U.S. Application Data
(63) Continuation-in-part of application No. 18/626,148, filed on Apr. 3, 2024.
Application No. 18/626,148 is a continuation-in-part of application No. 18/613,079, filed on Mar. 21, 2024.
Application No. 18/613,079 is a continuation-in-part of application No. 18/408,523, filed on Jan. 9, 2024.

(51) Int. Cl. A61N 5/06

(52) U.S. Cl. CPC A61N 5/0622; A61N 2005/0627; A61N 2005/0648; A61N 2005/0653; A61N 2005/0663

(57) ABSTRACT

A wearable light device includes a controller, a first light source, a second light source, and a wearable mechanism. The controller operates the first light source at a first frequency (F1)≥60 Hz and the second light source at a second frequency (F2), greater than F1. The wearable mechanism enables the device to fit on the head of a subject such that a first light emitted by the first light source is perceived by a first eye of the subject and a second light emitted by the second light source is perceived by the other eye of the subject. An invisible visual simulation at a frequency equal to F2-F1 is induced endogenously in the brain of the subject. The superimposed light of the first and second lights is flicker-free to eyes of a subject and can be used for treating or preventing Alzheimer's disease or memory degeneration.

The present disclosure is a continuation-in-part (CIP) of U.S. patent application Ser. No. 18/626,148 filed 3 Apr. 2024. Content of aforementioned application is herein incorporated by reference in its entirety.

### BACKGROUND

### Technical Field

The present disclosure pertains to the field of wearable light device and, more specifically, proposes a wearable light device with dual frequencies.

### Description of Related Art

It has been discovered that by flickering a light at a frequency between 35 Hz to 45 Hz or generating a sound at a similar frequency, it has the effect of stimulating the cells in certain regions of the brain, resulting in using a flicking light or a sound at such frequency for treating Alzheimer's disease. However, turning on and off a light source at a frequency between 35 Hz to 45 Hz can create visual discomfort for a subject. Different approaches have been introduced to overcome this visual discomfort under 40 Hz flickering light.

U.S. patent application Ser. No. 18/626,148 introduces a gamma stimulation apparatus that comprises a rectifier, a microcontroller, a first modulation operation switch (MOS), a second MOS, a first light source, and a second light source. The microcontroller sends the first MOS a first signal having a first periodical waveform at a first operating frequency (OF1), and the first MOS operates the first light source according to the first signal, producing a first light output at the OF1 frequency. The microcontroller sends the second MOS a second signal having a second periodical waveform at a second operating frequency (OF2), and the second MOS operates the second light source according to the second signal, producing a second light output at the OF2 frequency. The first light output and the second light output superimpose each other to form a superimposed light having a superimposed frequency equal to OF2-OF1, and the superimposed frequency is between 20 Hz and 45 Hz. The superimposed light appears flicker-free (free of flicker) to eyes of a subject.

The present disclosure extends the teaching in U.S. patent application Ser. No. 18/626,148 to a wearable light device with dual frequencies with a mechanism to fit on the head of a subject such that a light emitted by a first light source is perceived by a first eye of a subject and a second light emitted by the second light source is perceived by the other eye of the subject.

### SUMMARY

In one aspect, the wearable light device comprises a first light source, a second light source, a controller, and a wearable mechanism. The controller is configured to operate the first light source at a first frequency (F1)≥60 Hz, and the controller is configured to operate the second light source at a second frequency (F2), greater than F1. The wearable mechanism enables the device to fit on the head of a subject such that a first light emitted by the first light source is perceived by a first eye of a subject and a second light emitted by the second light source is perceived by the other eye of the subject. The subject must be able to view the first light at F1 frequency and the second light at F2 frequency simultaneously. This includes the situation where both eyes can perceive the first light and the second light at the same time, as well as the scenario where one eye can see the first light and the other eye can see the second light. Moreover, an invisible visual simulation at a frequency equal to F2-F1 is induced endogenously in the brain of the subject. When properly selecting F2 and F1, and thus their difference (F2-F1), the wearable light device may be used for treating Alzheimer's disease or memory degeneration. Given that F1 and F1 are both flicker-free, the subject cannot perceive any flickering from the wearable light device. Moreover, the device does not generate directly the differential frequency, F2-F1. Rather, the differential frequency is induced endogenously in the brain of the subject. Therefore, even though the differential frequency F2-F1 may be below 60 Hz, the subject still cannot perceive any flickering form the device.

The wearable light device is likely powered by an internal battery, or it may be powered by an external power source. There are various wearable mechanisms demonstrated by different electronic goggles on the market. Some use an elastic strap to fasten the goggles on the head of the subject. Some others take the shape of an eyeglass with a bridge fitting over the nose of the subject and two temples over the ears of the subject. Some others may use a wire frame to fit over the head of the subject.

In some embodiments, the first light is perceived only by the first eye of the subject and the second light is perceived only by the other eye of the subject. In other words, the first eye cannot perceive the second light, and the other eye cannot perceive the first light. Nonetheless, the subject can view the first light at F1 frequency (via the first eye) and the second light at F2 frequency (via the second eye) simultaneously. For example, the first light source fits over the left eye of the subject and the second light source fits over the right eye of the subject. Therefore, the left eye only perceives the first light whereas the right eye only perceives the second light. The brain of the subject can perceive a light stimulation endogenously at the frequency F2-F1.

In some embodiments, F2-F1 is between 35 Hz and 45 Hz. The light stimulation at a frequency between 35 Hz and 45 Hz is known to trigger gamma stimulation to certain regions of the brain and has the effect of improving Alzheimer's disease symptoms. Further in some embodiments, the F1 is 60 Hz and the F2 is 100 Hz. Further in some other embodiments, the F1 is 80 Hz and the F2 is 120 Hz.

In some embodiments, F2-F1 is between 3 Hz and 8 Hz. The light stimulation at a frequency between 3 Hz and 8 Hz is known to trigger theta stimulation to certain regions of the brain and has the effect of improving memory recall symptoms. In some embodiments, the F1 is 80 Hz, and the F2 may be 84 Hz or 87 Hz.

In some embodiments, the controller is configured to operate the first light source and the second light source for producing equal amount of light output (in lumen). By having both light sources each generating the same amount of light output, it reduces the perceivability of the flickering of the superimposed light of the first light and the second light. This is very useful since eyes are sensitive to the difference of light level.

In some embodiments, the first light source and the second light source are with a same color temperature (e.g., 4000K). Even though the eyes are less sensitive to the color temperature difference as compared to that of light level, having both light sources emitting the same color temperature reduces the perceivability of the flickering of the superimposed light.

In some embodiments, the first light source emits a wavelength between 380 nm and 780 nm, and the second light source emits a wavelength between 380 nm and 780 nm. In this case, the first light source and the second light source emit primarily visible wavelengths.

A recent study (https://journals.sagepub.com/doi/full/10.3233/JAD-220866) shows that near infrared (NIR) wavelengths also have positive effect in treating Alzheimer's disease. There may be situations where NIR wavelengths may be preferred since NIR wavelengths have additional health benefits such as improving blood circulation, lowering heartbeat, and improving the mood of the subject. Therefore, in some embodiments, the first light source emits a wavelength between 780 nm and 1100 nm, and the second light source emits a wavelength between 780 nm and 1100 nm, i.e., both in the NIR wavelength range. By using NIR wavelengths, the wearable light device provides the benefit of gamma or theta stimulation to the brain due to the simulating frequency F2-F1 as well as the benefits of NIR wavelengths such as improving blood circulation, lowering heartbeat, and improving the mood of the subject.

There are no restrictions on the technology of the first light source and the second light source, as long as they can operate at the F1 and the F2 accurately and effectively. In some embodiments, the first light source comprises a light emitting diode (LED) or organic LED (OLED), and the second light source comprises another LED or OLED.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts an exterior view of an embodiment of the present disclosure in the form of a wearable goggle.

FIG. 2 schematically depicts one layout of the first and the second light sources of the wearable goggle.

FIG. 3 schematically depicts another layout of the first and the second light sources of the wearable goggle.

FIG. 4 schematically depicts another embodiment of the present disclosure in the form of a pair of glasses wherein the light sources are LEDs.

FIG. 5 schematically depicts another embodiment of the present disclosure in the form of an attachable frame to a pair of glasses wherein the light sources are LEDs.

FIG. 6 schematically depicts another embodiment of the present disclosure in the form of a pair of glasses wherein the light sources are OLED displays.

FIG. 7 schematically depicts another embodiment of the present disclosure in the form of an attachable frame to a pair of glasses wherein the light sources are OLED display.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of wearable light devices having different form factors.

A wearable light device includes a controller, a first light source, a second light source, and a wearable mechanism. The controller operates the first light source at a first frequency (F1)≥60 Hz and the second light source at a second frequency (F2), greater than F1. The wearable mechanism enables the device to fit on the head of a subject such that a first light emitted by the first light source is perceived by a first eye of a subject and a second light emitted by the second light source is perceived by the other eye of the subject. An invisible visual simulation at a frequency equal to F2-F1 is induced endogenously in the brain of the subject. By carefully choosing F2 and F1, and thus their difference, the superimposed light of the first light and the second light is flicker-free to eyes of a subject and can be used for treating or preventing Alzheimer's disease or memory degeneration.

### Example Implementations

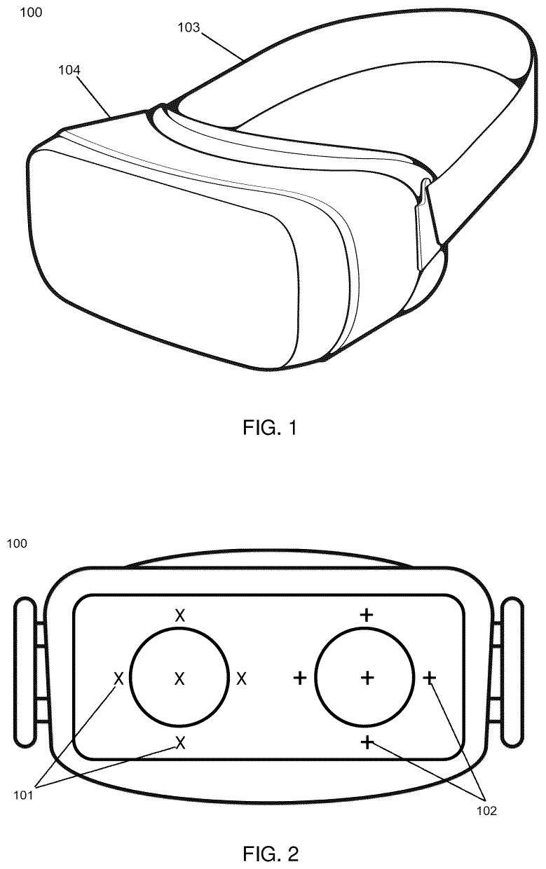

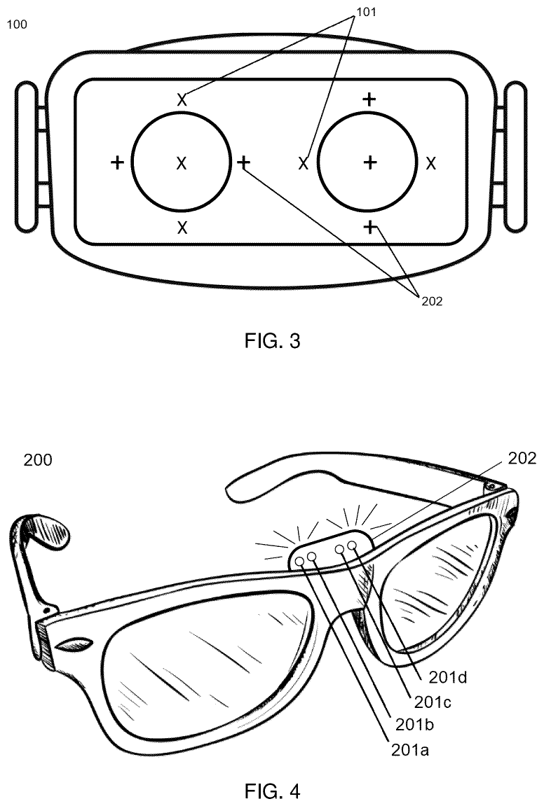

FIG. 1 shows an embodiment of the wearable light device of the present disclosure 100 in the form of an electronic goggle. The wearable mechanism is an elastic strap 103 that can fit over the head of a subject. FIG. 2 and FIG. 3 demonstrate two different layouts of the first light source 101 (denoted as “x” in the figures) and the second light source 102 (denoted at “+” in the figures). In FIG. 2, the first light source 101 shines to the left eye of the subject whereas the second light source 102 shines to the right eye of the subject. In FIG. 3, the first light source 101 and the second light source 102 shine to both eyes of the subject. The controller and a battery are hidden in the housing 104 of embodiment 100. The controller is configured to operate the first light source 101 at the F1 frequency 80 Hz and the second light source 102 at the F2 frequency 120 Hz. This creates a superimposed light at a differential frequency 40 Hz. Since both 80 Hz and 120 Hz are flicker-free to the subject, the superimposed light with the differential frequency equal to 40 Hz also appears flicker free to the subject. The light stimulation at 40 Hz frequency is known to trigger gamma stimulation to certain regions of the brain and has the effect of improving Alzheimer's disease symptoms.

In the case of FIG. 2, the left eye of the subject observes 80 Hz light which is flicker-free, and the right eye of the subject observes 120 Hz light which is also flicker-free. Nonetheless, an endogenous gamma stimulation at 40 Hz is created in the brain of the subject when the brain processes the light perceived by the left eye (at 80 Hz) and the light perceived by the right eye (at 120 Hz) simultaneously. Again, the subject observes no light flickering. The endogenous gamma stimulation in the brain at 40 Hz has the effect of improving Alzheimer's disease symptoms.

The differential frequency at 40 Hz is chosen for treating or preventing Alzheimer's disease. If the F2 frequency for the second light source 102 changes to 84 Hz, then the differential frequency becomes 4 Hz. This induces an endogenous theta stimulation at 4 hz for treating memory degeneration.

The first light source 101 and the second light source 102 both comprise the same 4000K (color temperature) LED. The controller operates both light sources to produce equal amounts of light output. Alternatively, the first light source and the second light source may comprise the same NIR LED emitting 850 nm wavelength, for additional health benefits such as improving blood circulation, lowering heartbeat, and improving the mood of the subject.

FIG. 4 shows another embodiment of the present disclosure in the form of a pair of glasses 200. There are four LED light sources 201a, 201b, 2010, 201d that emit a same amount of light output at a same color temperature, e.g., 4000K. The controller and a battery are hidden in the housing 202 of embodiment 200. The controller operates light sources 201a and 201b at 80 Hz and the light sources 201c and 201d at 120 Hz. Alternatively, the controller operates light sources 201a and 201c at 80 Hz and the light sources 201b and 201d at 120 Hz.

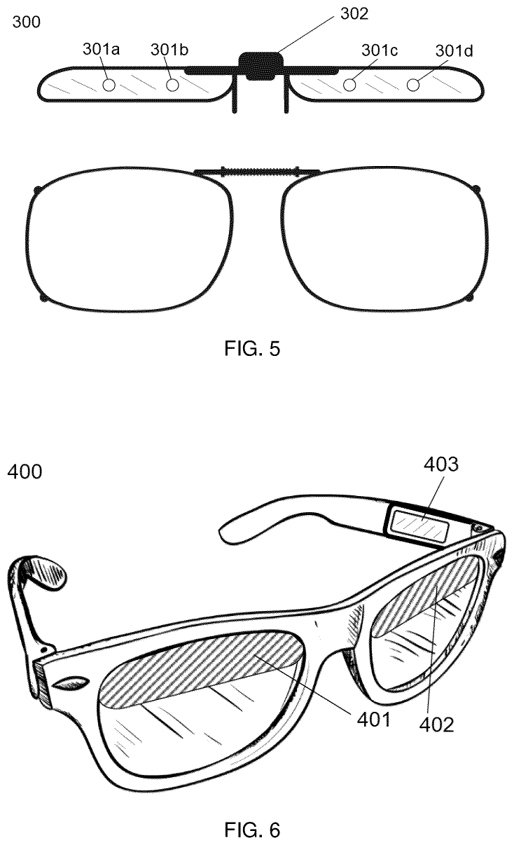

FIG. 5 shows another embodiment of the present disclosure in the form of an attachable frame 300 that can be attached to a pair of glasses. There are four LED light sources 301a, 301b, 301c, 301d that emit a same amount of light output at a same color temperature 4000K. The controller and a battery are hidden in the housing 302 of embodiment 300. The controller operates light sources 301a and 301b at 80 Hz and the light sources 301c and 301d at 120 Hz. Alternatively, the controller operates light sources 301a and 301c at 80 Hz and the light sources 301b and 301d at 120 Hz.

FIG. 6 shows another embodiment of the present disclosure in the form of a pair of glasses 400. There are two OLED displays 401 and 402 as the two light sources. The two OLED displays 401 and 402 are overlaid on the top portion of the two lenses of the glasses. Two OLED displays 401, 402 each may emit a same or a different amount of light output, and they each may emit a same or a different color temperature. The controller and a battery are hidden in the housing 403 of embodiment 400. The controller operates the OLED display 401 (the first light source) at 80 Hz and the OLED display 402 (the second light source) at 120 Hz.

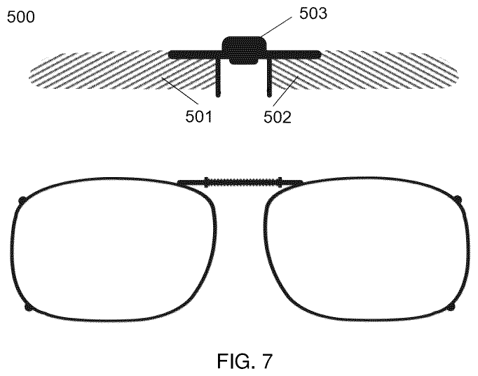

FIG. 7 shows another embodiment of the present disclosure in the form of an attachable frame 500 that can be attached to a pair of glasses. There are two OLED displays 501 and 502 are two light sources: one on the left of the attachable frame and the other on the right. Two OLED displays 501, 502 each may emit a same or a different amount of light output, and they each may emit a same or a different color temperature. The controller and a battery are hidden in the housing 503 of embodiment 500. The controller operates the OLED display 501 (the first light source) at 80 Hz and the OLED display 502 (the second light source) at 120 Hz.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A wearable light device, comprising:
a first light source;
a second light source;
a controller;
a wearable mechanism, and
wherein:
the controller is configured to operate the first light source at a first frequency (F1)≥60 Hz,
the controller is configured to operate the second light source at a second frequency (F2) greater than F1,
the wearable mechanism enables the device to fit on a head of a subject such that a first light emitted by the first light source is perceived by a first eye of the subject and a second light emitted by the second light source is perceived by a second eye of the subject, and
an invisible visual simulation at a frequency equal to F2-F1 is induced endogenously in a brain of the subject.

2. The wearable light device of claim 1, wherein the first light is perceived only by the first eye of the subject and the second light is perceived only by the second eye of the subject.

3. The wearable light device of claim 1, wherein F2-F1 is between 35 Hz and 45 Hz.

4. The wearable light device of claim 1, wherein the F1 is 60 Hz and the F2 is 100 Hz.

5. The wearable light device of claim 1, wherein the F1 is 80 Hz and the F2 is 120 Hz.

6. The wearable light device of claim 1, wherein F2-F1 is between 3 Hz and 8 Hz.

7. The wearable light device of claim 1, wherein the controller is configured to operate the first light source and the second light source to each produce an equal amount of light output in lumen.

8. The wearable light device of claim 1, wherein the first light source and the second light source are with a same color temperature.

9. The wearable light device of claim 1, wherein the first light source emits a first wavelength between 380 nm and 780 nm, and wherein the second light source emits a second wavelength between 380 nm and 780 nm.

10. The wearable light device of claim 1, wherein the first light source emits a first wavelength between 780 nm and 1100 nm, and wherein the second light source emits a second wavelength between 780 nm and 1100 nm.

11. The wearable light device of claim 1, wherein the first light source comprises a light emitting diode (LED) or organic LED (OLED), and wherein the second light source comprises another LED or OLED.
